(19) United States
(12) Patent Application Publication
(10) Pub. No.: US 2024/0382602 A1
(43) Pub. Date: Nov. 21, 2024

(54) A CIS-PLATINUM(II)-OLIGOMER HYBRID

(71) Applicant: Dublin City University, Dublin (IE)

(72) Inventors: Andrew Kellett, Dublin (IE); Joseph Hennessy, Dublin (IE)

(21) Appl. No.: 18/694,047

(22) PCT Filed: Sep. 22, 2022
(86) PCT No.: PCT/EP2022/076459

§ 371 Date: Mar. 21, 2024

(30) Foreign Application Priority Data
Sep. 22, 2021 (GB) 2113541.3

(51) Int. Cl. A61K 47/54

(52) U.S. Cl. CPC A61K 47/549

(57) ABSTRACT

A cis-platinum(II)-oligomer hybrid capable of targeting purine-rich targets in genomic DNA, and crosslinking the DNA, is described. The hybrids are generated by conjugating an azide-modified cis-platinum(II) complex with an alkyne-modified monomer of an oligomer by azide-alkyne cycloaddition, in which the oligomer comprises at least 10 contiguous nucleobase-bearing monomers. The oligomer may be a triplex-forming oligonucleotide. Methods of treating proliferative disorder such as cancer are also described.

### FIELD OF THE INVENTION

The present invention relates to a cis-platinum(II)-oligomer hybrid. In particular the invention relates to a cis-platinum(II)-oligonucleotide hybrid. The invention also relates to the use of the hybrids of the invention to treat cancer in a mammal.

### BACKGROUND TO THE INVENTION

Therapies that target the downstream inhibition of gene expression are of great current research significance with considerable efforts now dedicated to the discovery of new antisense oligonucleotides (ASOs). [1] The development of nucleic acid probes targeting DNA, such as triplex-forming oligonucleotides (TFOs), [2] offers an alternative strategy whereby gene expression is directly inhibited at the genomic level. Here, we present a click chemistry strategy for the generation of a new class of cis-platinum(II)-TFO hybrid biomaterial that possess DNA binding and crosslinking activity. Homopyrimidine TFOs (containing T and C bases only) bind non-covalently in the major groove of duplex DNA to purine-rich target strands through Hoogsteen hydrogen bond formation with AT and GC base pairs producing T-AT and C⁺-GC parallel triplex motifs. [3].

One of the main challenges confronting the application of TFOs is their weak duplex binding affinity and poor in vitro and in vivo stability. [4] Recent work has therefore focused on enhancing target binding properties and maximising their lifetimes in cellular environments. As part of this effort, the introduction of a covalent crosslinking agent such as psoralen into TFO constructs has shown significant promise (FIG. 1). [5] Here, crosslinking is initiated by UV light [6] to produce gene-specific mutations [7] that inhibit transcriptional activity. [8] Alternatively, the use of TFOs as targeting probes that discretely transport platinum(II) crosslinking agents to selected duplex targets has also been explored. [9] Several motifs exist but sustained efforts to coordinate trans-Pt(II) complexes—such as trans-dichlorodiammineplatinum(II)—to a guanine (mismatch) base within TFO constructs has enabled the generation of mono-functional crosslinking hybrids (FIG. 1). [10] Here, a single labile trans site on the probe-bound Pt(II) complex is available to crosslink with a purine base on the target duplex. Although monofunctional Pt(II)-TFOs have previously been generated through platinum-derivatised phosphoramidites, [11] Surprisingly little research has been carried out to extend this concept towards the construction of bifunctional cisplatin(II)-TFO motifs. Attempts to incorporate platinated nucleotide building blocks into oligonucleotides (ON) using solid-phase synthesis have yielded inactive platinum products, [12] although a cis-platinum(II) tethered ON was shown to retain crosslinking ability and displayed adduct formation against a 25:29 mer duplex target. [13] While many Pt(II) and Pt(IV) complexes have been developed that overcome cisplatin cross-resistance while maintaining a comparable cytotoxic effect, [14-16] the critical issue of lack of nucleic acid target specificity underpins the requirement for the development of alternative strategies to enhance platinum-based compounds target specificity and selectivity.

It is an objective of the invention to overcome at least one of the above referenced problems.

### SUMMARY OF THE INVENTION

The objective is met by the provision of a click chemistry-based approach that combines alkyne-modified oligomers (for example triplex-forming oligonucleotides) with azide-bearing cis-platinum(II) complexes—generally based on cisplatin, oxaliplatin, and carboplatin type motifs—to generate a library of Pt(II)-oligomer hybrids. These constructs can be assembled modularly and enable directed platinum(II) crosslinking to nucleic acids, including purine-rich genomic target sequences, and single stranded DNA and RNA, under the guidance of the oligomer probe. Azide groups are incorporated into cis-platinum(II) scaffolds to afford suitable handles for click chemistry coupling with alkyne-modified TFOs. In order to maximise their crosslinking potential, platinum(II) complexes are generally designed to be geometrically and structurally similar to clinical agents cisplatin, carboplatin, and oxaliplatin. To demonstrate the invention, purine-rich tracts of the green fluorescent protein (GFP) gene are targeted and Pt-TFO hybrid stability and crosslinking are assessed through thermal melting studies and native and denaturing PAGE analysis.

In comparison to existing methods used to prepare platinated TFOs, click chemistry affords a modular approach whereby the incorporation of cis-platinum(II) at practically any location within the probe strand is possible. The invention also overcomes limitations requiring complexation between a platinum(II) reagent, such as a trans-platinum(II) complex, [10a, 10b] and the TFO substrate—an approach that hitherto precluded the development of cis-platinum(II) type hybrids [13a] and the ability to generate 1,2-d(GpG) cisplatin lesions central to their clinical success.

In a first aspect, the invention provides a cis-platinum(II)-oligomer hybrid in which the oligomer comprises at least 10 (generally contiguous) nucleobase-bearing monomers. Typically the oligomer is generated by conjugating an azide-modified cis-platinum(II) complex with an alkyne-modified monomer of an oligomer by azide-alkyne cycloaddition.

In any embodiment, the oligomer is an oligonucleotide. In other embodiments, the oligomer may be a nucleic acid variant such as a peptide nucleic acid (PNA), locked nucleic acid (LNA), phosphorodiamidate morpholino oligomer (PMO), phosphorothioate (PS), 2′-modified PS such as 2′-O-methoxyethyl (2′-MOE) and PS 2′-constrained ethyl (2′-cEt). All of these variants comprise monomers having a nucleobase and a connecting group.

In any embodiment, the oligonucleotide is a triplex-forming oligonucleotide.

In any embodiment, the azide-modified cis-platinum(II) complex is a compound of Formula (I):

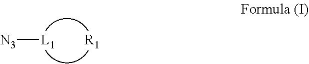

in which:
L₁ is a linker;
R₁ is a platinum containing DNA binding fragment of a cis-platinum (II) complex;
and L₁ binds to R₁ via bidentate coordination.

In any embodiment, L₁ binds to R₁ via bidentate coordination selected from: diam(m)ine; disulphate; N,O; N,S; O,S; or O,O′ bidentate coordination.

The linker L₁ may be any group capable of conjugating an azide group to a cis-platinum(II) complex, for example a straight or branched, substituted or unsubstituted, aryl group, including a lower alkyl or lower alkoxy group. In one embodiment, L₁ is a branched lower alkyl group.

In any embodiment, the azide-modified cis-platinum(II) complex is selected from:

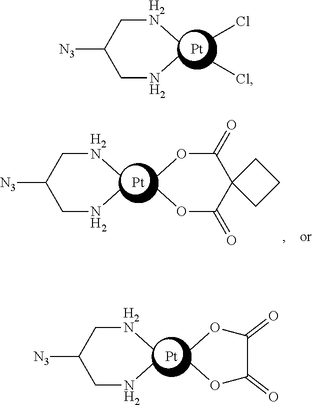

In any embodiment, the alkyne modified monomer of the oligomer comprises an alkyne substituent conjugated to the nucleobase of the monomer.

In any embodiment, the nucleobase of the alkyne modified monomer is selected from:

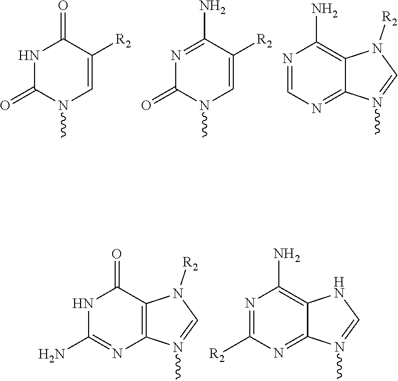

in which:
R₂ is selected from C (CH), L₂-C(CH) and a cycloalkyne substituent, in which L₂ is a linker.

The linker L₂ may be any group capable of conjugating an alkyne group to a carbon or nitrogen atom of a purine or pyrimidine nucleobase for example a straight or branched, substituted or unsubstituted, linking group such as an aryl group, in particular a lower alkyl or lower alkoxy group.

In any embodiment, the monomers of the oligomer include a ribose, in which the alkyne modified monomer of the oligomer comprises an alkyne substituent conjugated to the 5′ phosphate of a ribose of the monomer.

In any embodiment, the oligomer comprises a phosphate deoxyribose backbone, in which the alkyne modified monomer of the oligomer comprises an alkyne substituent conjugated to the 5′ phosphate of a ribose of the monomer.

In any embodiment, the oligomer is an oligonucleotide, and in which the alkyne modified monomer of the oligonucleotide comprises an alkyne substituent conjugated to the 5′ phosphate of a ribose of the monomer.

In any embodiment, the alkyne substituent is a cycloalkyne substituent selected from:

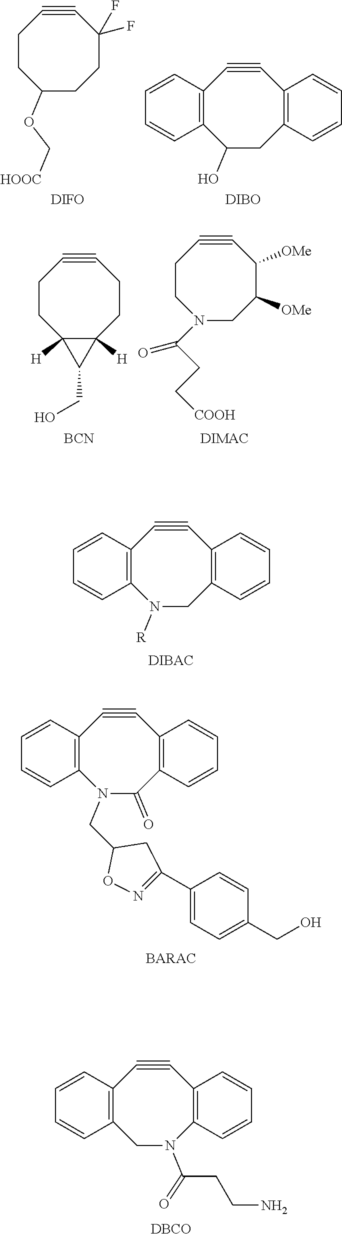

In DIBAC, R may be a reactive functional group for coupling to a monomer of the oligomer, for example a hydroxyl, carboxyl, or amine group, and optionally including a linker.

In any embodiment, the azide-alkyne cycloaddition is selected from metal-catalysed azide-alkyne cycloaddition and strain promoted azide-alkyne cycloaddition (SPAAC).

In any embodiment, the metal-catalysed azide-alkyne cycloaddition is copper(I) catalysed azide-alkyne cycloaddition (CuAAC). Other metal catalysts may be employed, including ruthenium or silver.

In any embodiment, the oligomer is a triplex forming oligonucleotide in which at least 70% of the monomers of the triplex forming oligonucleotide comprise a pyrimidine nucleobase.

In any embodiment, the oligomer comprises 10 to 30 monomers.

In any embodiment, the alkyne modified monomer of the oligomer is located internally or at the 5′ end of the oligomer.

In any embodiment, the cis-platinum(II)-oligomer hybrid has a structure selected from Formula (II) or (III):

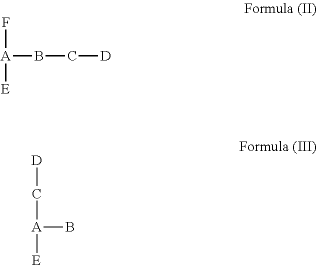

in which the oligomer comprises at least 10 contiguous nucleobase-bearing monomers, and in which:
A is a linker;
B is a purine or pyrimidine base; cis-platinum(II) complex;
A-B is a monomer of the oligomer;
C is a linker comprising a triazole or bicyclic triazole group formed by alkyne-azide cycloaddition;
D is a cis-platinum (II) complex;
E is part of the oligomer; and
F is absent or is part of the oligomer.

In Formula (II), the cis-platinum (II) modified monomer is located internally in the oligomer, and F comprises at least one monomer (e.g. 1 to 10 monomers) and E comprises at least one monomer (e.g. 1 to 10) such that the oligomer comprises at least 10, 12 or 15 monomers.

In Formula (III), the cis-platinum (II) modified monomer is located at the 5′ end of the oligomer, and E comprises at least monomers such that the oligomer comprises at least 10, 12 or 15 monomers.

The linker A is a connecting group of the oligomer. When the oligomer is an oligonucleotide, A is generally a phosphate deoxyribose or ribose group. When the oligomer is a PNA, A is usually N-(2-aminoethyl)-glycine. When the oligomer is LNA, A is usually phosphate or phosphorothioate 2′-4′ bridged ribose. When the oligomer is PMO, A is usually a morpholino group with phosphorodiamidate linkages.

In any embodiment, A is a phosphate deoxyribose unit.

In any embodiment, the oligomer is an oligonucleotide.

In any embodiment, the oligonucleotide is a triplex forming oligonucleotide.

In any embodiment, F-A-E in Formula (II) has a structure selected from Formula (IV) or (V) in which E and F are as defined previously:

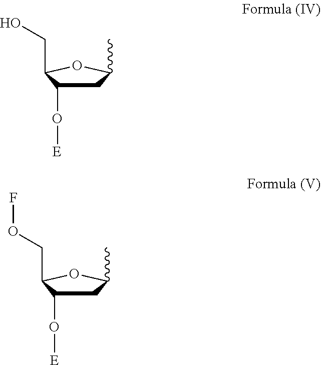

In any embodiment, in Formula (II), -B-C-D has a structure selected from Formula (VI), (VII), (VIII), (IX), or (X):

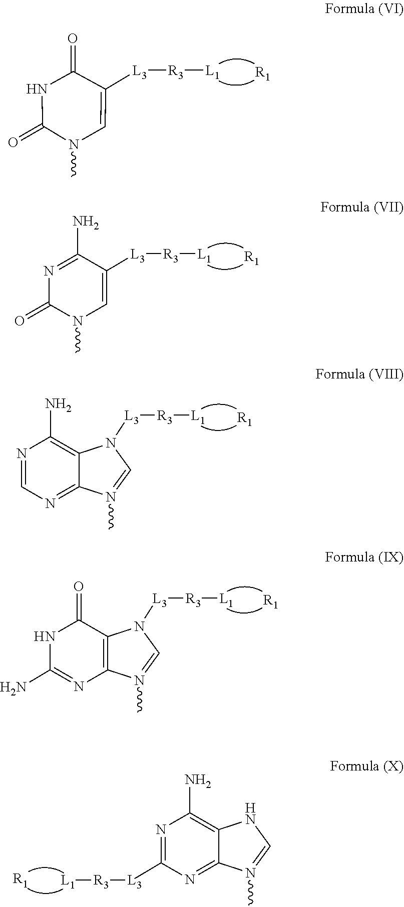

in which:
L₁ and L₃ are each, independently, a linker and in which L₃ may be absent;
R₃ is selected from a 1,4 substituted triazole, 1,5 substituted triazole group, or a strain promoted cycloalkane group;
R₁ is a platinum containing DNA binding fragment of a cis-platinum(II) complex; and L₁ binds to R₁ via bidentate coordination.

In any embodiment, L₃ is absent or is (CH₂)ₙ in which n is selected from 1 to 10.

In any embodiment, L₃ comprises a tertiary amine.

In any embodiment, L₃ has a structure of Formula (XI):

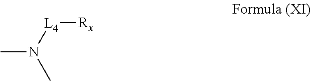

in which:
L₄ is a linker; and
Rₓ is an intercalator.

In any embodiment, Rₓ is selected 1-naphtoic acid, anthracene-9-carboxylic acid, phenanthrene-9-carboxylic acid, pyrene-1-carboxylic acid, quinoline-5-carboxylic acid, acridine-9-carboxylic acid, benzoacridine-12-carboxylic acid, benzophenanthridine-6-carboxylic acid, 1,10-phenanthroline-5-carboxylic acid, 6-aminophenanthridine, phenanthridine-6-carboxylic acid, thiazole orange-B6, or thiazole orange-Q6.

In any embodiment, L₄ has a structure of Formula (XII)

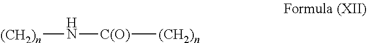

in which each occurrence of n is, independently, 0 to 10.

In any embodiment, the cis-platinum (II)-triplex forming oligonucleotide of the invention has a structure of Formula (XIII):

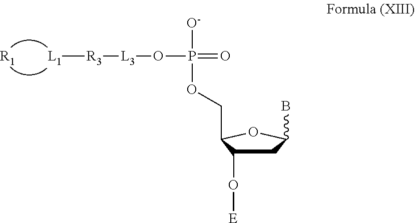

in which: L₁ and L₃ are each, independently, a linker and in which L₃ may be absent;
R₃ is selected from a 1,4 substituted triazole, or 1,5 substituted triazole group, or a strain promoted cycloalkane group;
R₁ is a platinum containing DNA binding fragment of a cis-platinum(II) complex; and
L₁ binds to R₁ via bidentate coordination.

In any embodiment, the cis-platinum(II)-oligomer of the invention is selected from:

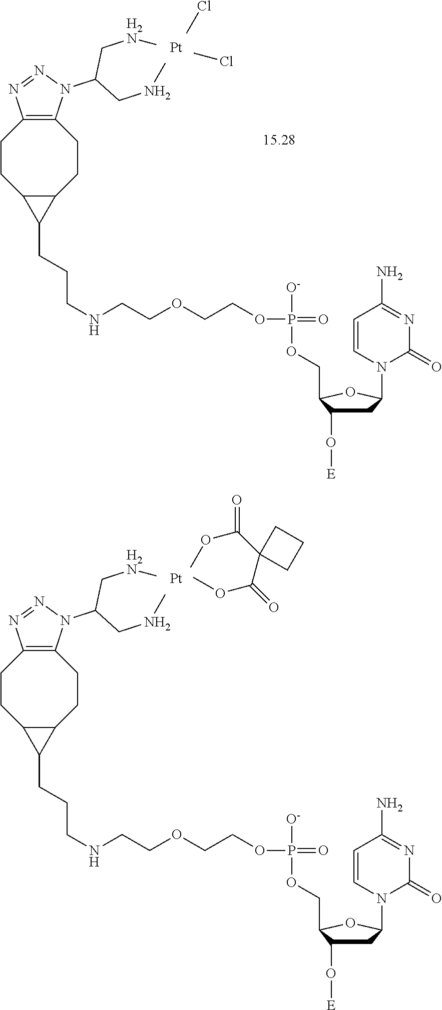

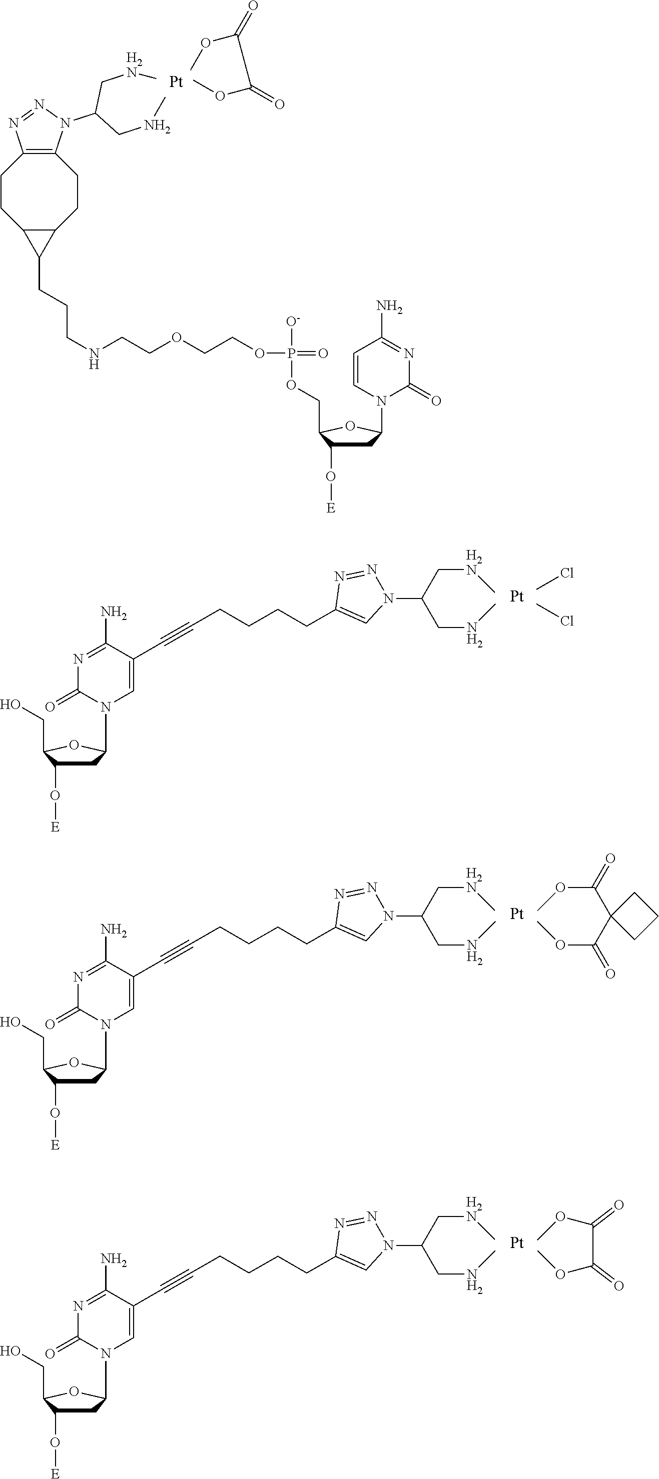

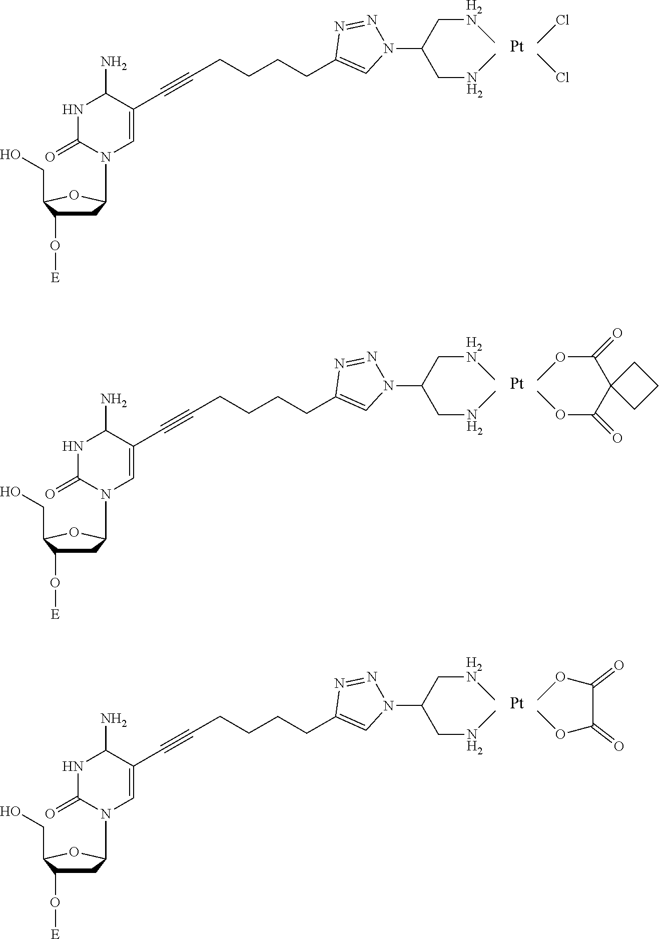

In any embodiment, the cis-platinum(II)-oligomer of the invention is selected from:

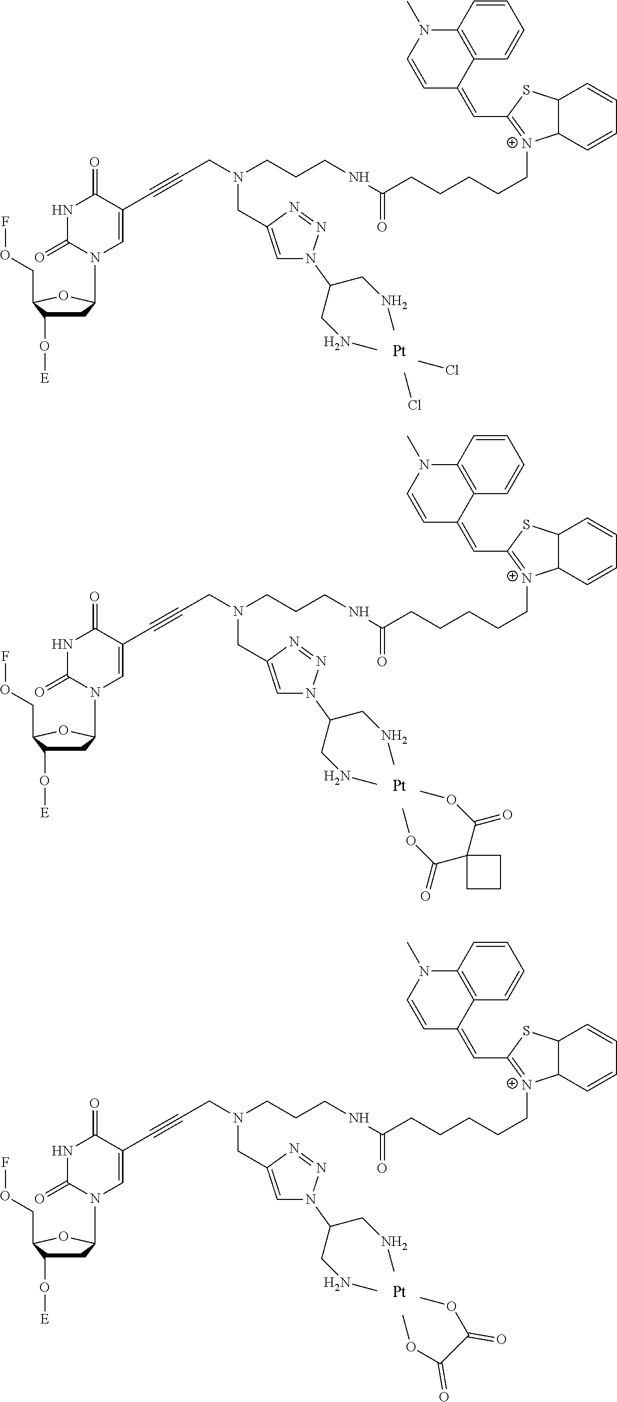

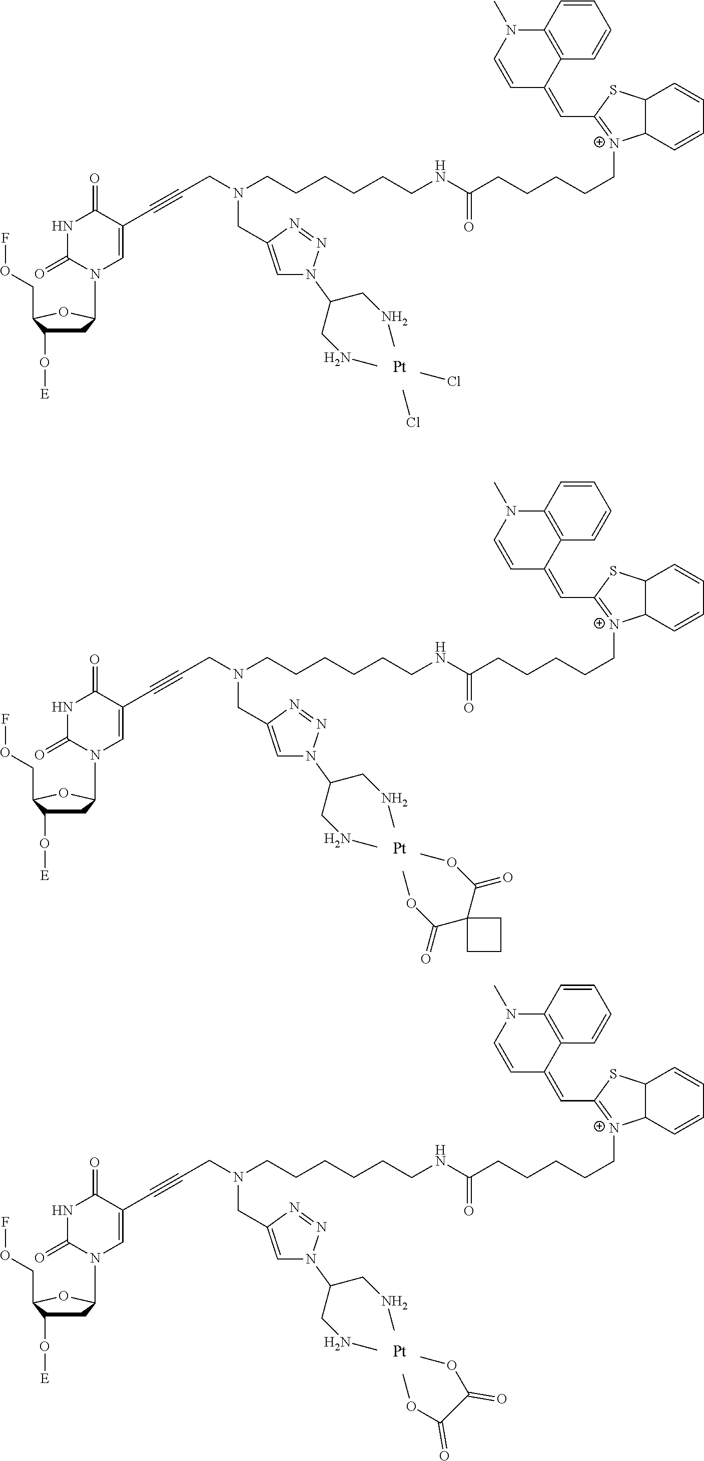

The invention also relates to a pharmaceutically acceptable salt of the hybrid of the invention.

In another aspect, the invention provides a pharmaceutical composition comprising a cis-platinum(II) oligomer hybrid of the invention and a pharmaceutically acceptable carrier

In another aspect, the invention provides a cis-platinum(II) oligomer hybrid, or composition, of the invention, for use in therapy, for use as a medicament, for use in the treatment or prevention of cancer, or for use in gene therapy. Other aspects and preferred embodiments of the invention are defined and described in the other claims set out below.

### BRIEF DESCRIPTION OF THE FIGURES

FIG. 1 (Prior Art). Current crosslinking hybrid constructs.

FIG. 2. Overview of Pt(II)-TFO hybrid design and application. Development of azide bearing platinum(II) complex prior to click chemistry generation of Pt(II)-TFO hybrids. Triplex formation is obtained through parallel Hoogsteen binding to the target dsDNA. Crosslink formation with the duplex target is facilitated by the triplex formation.

FIG. 3: Synthetic routes for the preparation of azide-functionalised platinum(II) complexes. Route 1. Preparation of cis-[Pt(2-azidopropane-1,3-diamine)Cl2] (Pt-N₃-Cis, 5, 27%). Route 2. Preparation of cis-[Pt(2-azidopropane-1,3-diamine)(CBDCA)] (Pt-N₃-Carbo, 7, 36%) and cis-[Pt(2-azidopropane-1,3-diamine)(Oxalate)] (Pt-N₃-Oxali, 8, 72%)

FIG. 4: A. Alkyne-modified nucleobases. Click combination with each of the azide-functionalised platinum(II) complexes. B. TFO sequences with nucleobase modifications denoted and specific recognition sites indicated within the respective duplex target.

FIG. 5. A. dsDNA sequences of the GFP gene with triplex recognition site in red.B. ΔTM of target dsDNA sequences treated with 2.5 eq. of azide-functionalised platinum(II) complexes. C. Specific TM values for duplex sequences when treated with Pt-N₃-Cis, Pt-N₃-Carbo or Pt-N₃-Oxali respectively.

FIG. 6. A. TM values of triplex melting observed for TFO3 and TOTFO hybrids. B. Thermal melting isotherms showing destabilising and stabilising effects in comparison to alkyne-modified TFO3. Key: ⋅=TFO3-Carbo; ⋅=Alkyne-TFO3; ⋅=TOTFO1-Carbo; and ⋅=TOTFO2-Carbo represent the respective mid-points of the triplex transitions.

FIG. 7. A. TFO3 with modified internal octadinyl-dU nucleobase and respective GFP target sequence. B. TFO3-Cis hybrid triplex formation with higher band structures observable. C. TFO3-Carbo hybrid. Triplex formation is significant even at lower loadings. D. TFO3-Oxali hybrid with higher band triplex formation—slower rate of triplex formation in comparison with TFO3-Carbo.

FIG. 8. A. TFO3-Cis hybrid and fluorescently labelled Duplex 3. B. NaCN treatment of unmodified triplex system. C. NaCN (5,000 eq.) afforded reversal of Pt-triplex system formed by TFO3-Cis hybrid.

FIG. 9. A. Denaturing PAGE experimental design. B. Terminal and internal nucleobase modification octadinyl-dU and azide-functionalised Pt-N₃-Carbo. C. Modified TFO sequences and dsDNA targets. D. Denaturing PAGE analysis: TFO-hybrids (50 eq.) were incubated at 37° C. with duplex targets for a minimum of 48 hours prior to electrophoresis. Upper band (Lanes 3-6) under Cy5 filter is indicative of adduct formation with purine strand. No adduct formation observed for TFO-17C (Lanes 7 and 8).

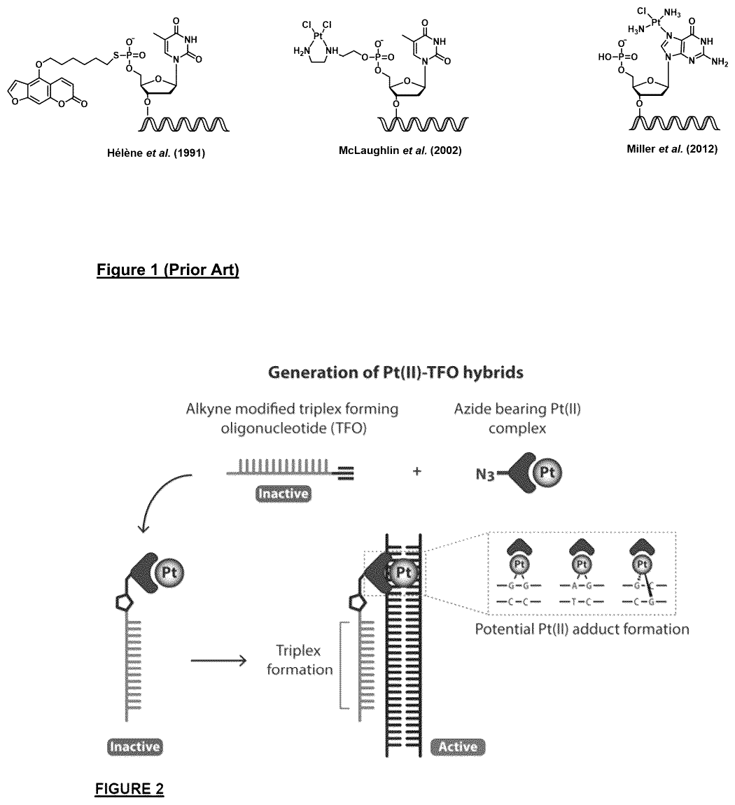

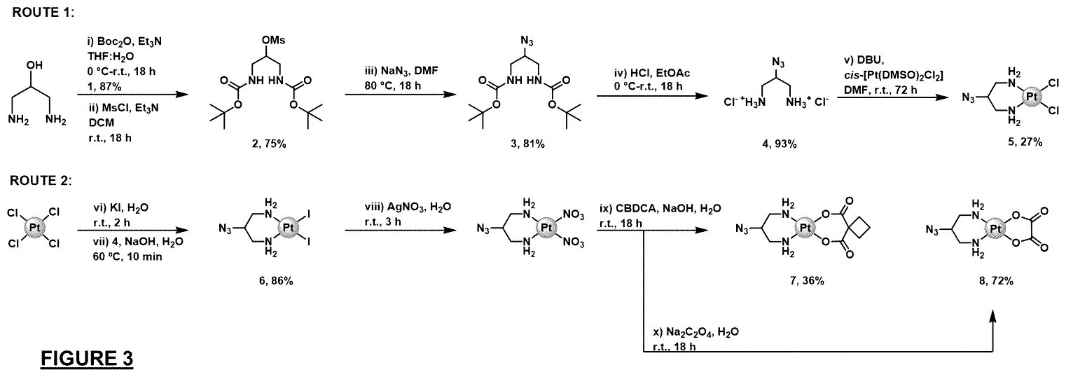

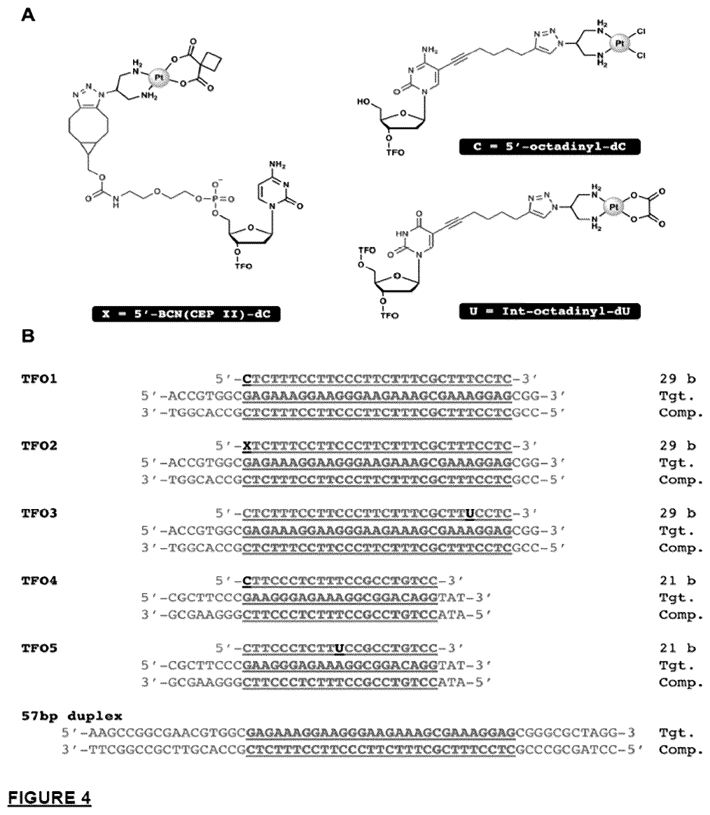

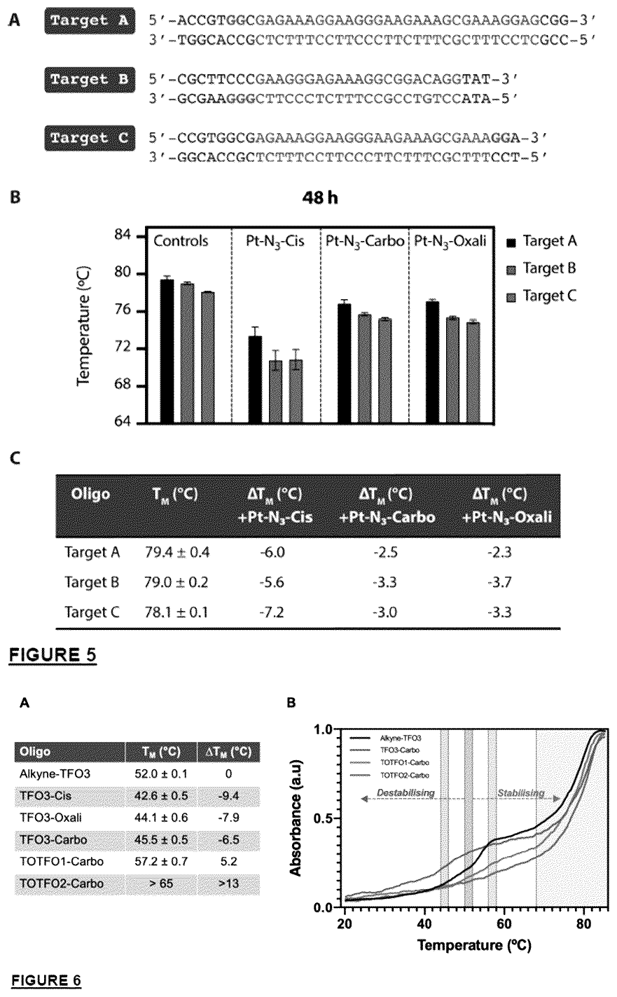

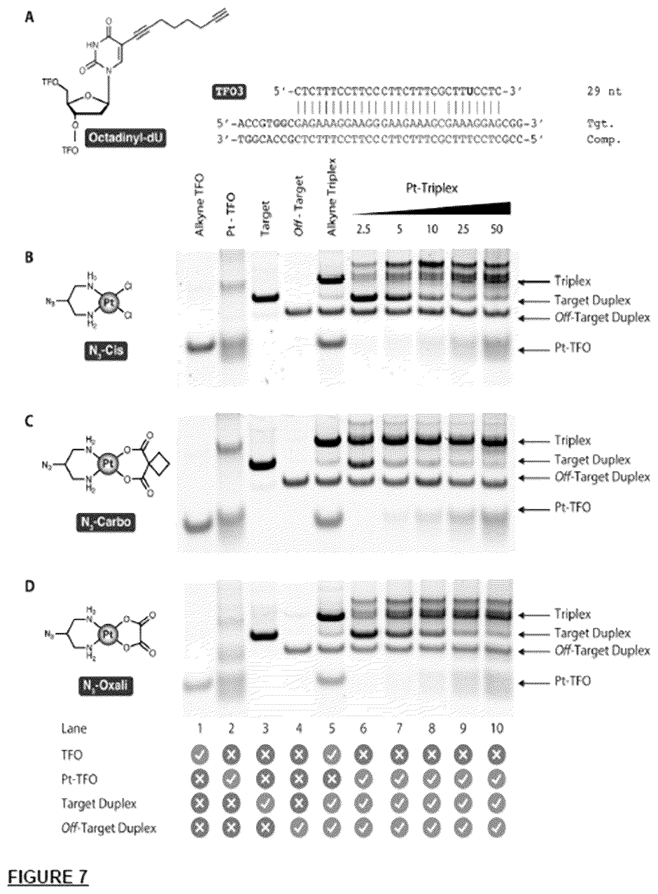

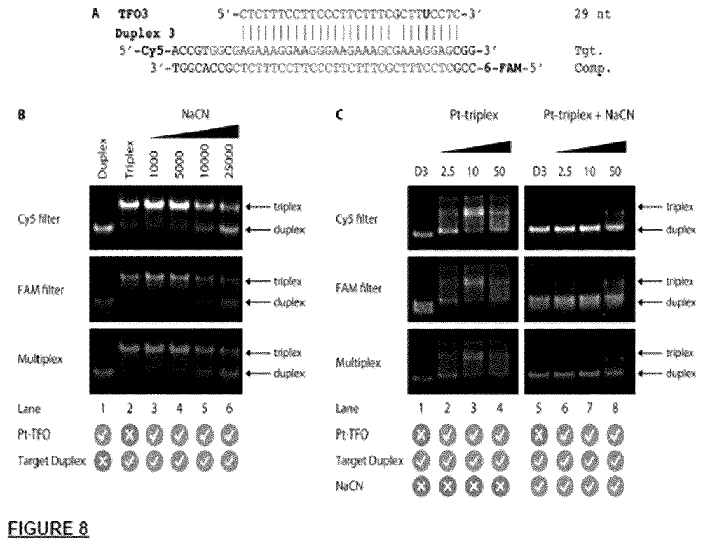

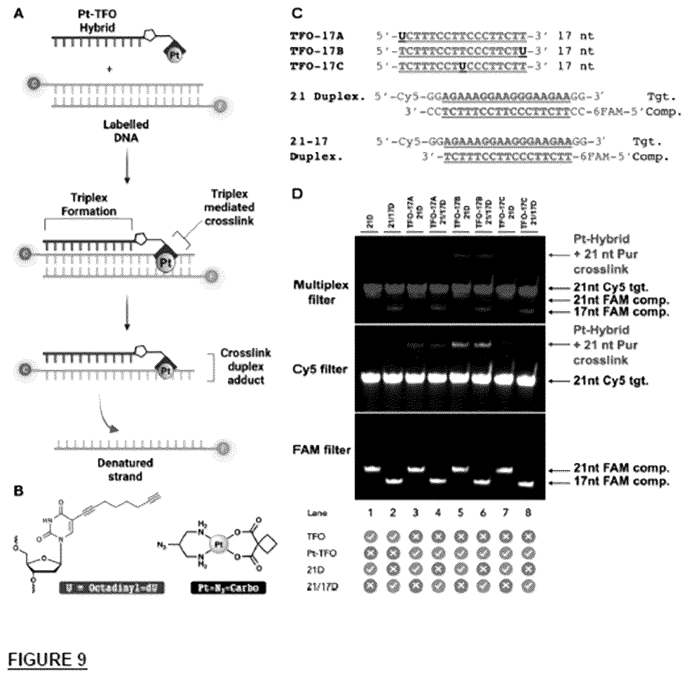

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

The term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The term “cis-platinum(II)-oligomer hybrid” refers to an oligomer comprising at least ten monomers linked together in which each monomer comprises a nucleobase and in which at least one of the monomers is covalently bound to a cis-platinum(II) complex that is capable of crosslinking a nucleic acid such as DNA or RNA. The oligomer is generally at least 10 or 12 monomers in length, for example 10-50, 10-40, 10-30, 12-40, 12-30, 15 to 40, 15 to 30, 20-40 or 20-30 monomers. The oligomer may be an oligonucleotide, or an oligonucleotide like variant such as for example a peptide nucleic acid (PNA) [17], locked nucleic acid (LNA) [18], phosphorodiamidate morpholino oligomer (PMO) [19], phosphorothioate (PS) [20], 2′-modified PS such as 2′-O-methoxyethyl (2′-MOE) [21] and PS 2′-constrained ethyl (2′-cEt). [22] in which each monomer comprises a nucleobase linked together by connecting groups to form a nucleic-acid like oligomer. The cis-platinum(II) complex is generally conjugated to the nucleobase on one of the monomers via a triazole or bicyclic triazole group (examples of which are provided herein). Such a linking group is formed as a result of alkyne-azide cycloaddition chemistry. The cis-platinum(II) complex may also be conjugated to another part of a monomer, for example the 5′ phosphate group of a nucleotide (when the oligomer is an oligonucleotide) or via a connecting group of a nucleic acid variant such as a ribose or peptide linker. Generally, only one of the monomers of the oligomer is functionalized with a cis-platinum(II) complex. The cis-platinum(II) complex may be conjugated to a 5′ monomer of the oligomer or to an internal monomer in the oligomer. The oligomer generally includes at least two types of nucleobases, for example two purines or two pyrimidines, or both purine and pyrimidine. In some cases, the oligomer may be predominantly formed or purines or pyrimidines, for example it may be constituted by at least 60%, 70%, 80%, 90% or 100% purines or pyrimidines. Generally, the oligomer is designed to preferentially bind to a target sequence, for example a double stranded nucleic acid molecule such as a sequence of genomic DNA or a single stranded nucleic acid such as single stranded DNA or RNA. In one preferred embodiment, the oligomer is a triplex-forming oligonucleotide. Other target sequences include ssDNA, mRNA, rRNA, tRNA, snRNA, cRNA, and ncRNA.

The term “cis-platinum(II)-complex” refers to a platinum-containing complex having a cis geometry and comprising a leaving group(s) that is capable of crosslinking a nucleic acid such as DNA or RNA. The crosslink may be interstrand or intrastrand crosslinking. Examples include Cisplatin, Oxaliplatin and Carboplatin, and other classical cis-platinum(II) complexes are described in the literature, for example Johnstone et al. Chem. Rev. 2016, 116, 5, 3436-348, Feb. 11, 2016 https://doi.org/10.1021/acs.chemrev.5b00597.

The term “azide-alkyne cycloaddition” as applied herein refers to a method of conjugating a synthetic biomolecule with a cis-platinum(II) complex by click chemistry to form a hybrid of the invention. The term includes metal-catalysed azide-alkyne cycloaddition (MCAAC), especially copper(I)-catalysed azide-alkyne cycloaddition (CuAAC), and strain promoted azide-alkyne cycloaddition (SPAAC). Azide-alkyne cycloaddition is described in the literature, for example by Huisgen. Angew. Chem. Int. Ed. 1963, 2 (10), 565-598. https://doi.org/10.1002/anie.196305651. Sharpless et al. Angew. Chem. Int. Ed. 2001, 40 (11), 2004-2021. https://doi.org/10.1002/1521 3773(20010601)40:11%3C2004:AID-ANIE2004%3E3.0.CO; 2-5 and Meldal et al. J. Org. Chem. 2002, 67(9), 3057-3064. https://doi.org/10.1021/jo011148j. Methods of forming hybrid molecules by CuAAC and SPAAC are described herein.

The term “cycloalkyne substituent” as applied herein refers to a cycloalkyne containing substituent that incorporates a reactive functional group for coupling to a monomer of the oligomer in which the cycloalkyne group is capable of reacting with an azide group of an azide-modified cis-platinum(II) complex to form the hybrids of the invention. Examples of cycloalkyne substituents are provided herein.

The term “triplex-forming oligonucleotide” or “TFO” refers to an oligonucleotide which bind as third strands to duplex DNA in a sequence specific manner. The oligonucleotides are synthetic or isolated nucleic acid molecules which selectively bind to or hybridize with a predetermined target sequence, target region, or target site within or adjacent to a human gene so as to form a triple-stranded structure. Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful. The oligonucleotides are preferably generated using known DNA synthesis procedures. In one embodiment, oligonucleotides are generated synthetically. Oligonucleotides can also be chemically modified using standard methods that are well known in the art. The nucleotide sequence of the oligonucleotides is selected based on the sequence of the target sequence, the physical constraints imposed by the need to achieve binding of the oligonucleotide within the major groove of the target region, and the need to have a low dissociation constant (K<j) for the oligonucleotide/target sequence. The oligonucleotides have a base composition which is conducive to triple-helix formation and is generated based on one of the known structural motifs for third strand binding. The most stable complexes are formed on polypurine: polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C<+>.G:C and T.A:T. The triplex structures are stabilized by two

Hoogsteen hydrogen bonds between the bases in the TFO strand and the purine strand in the duplex. A review of base compositions for third strand binding oligonucleotides is provided in U.S. Pat. No. 5,422,251. Considerations when formulating a TFO are described in WO2017143042.

“Alkyl” refers to a group containing from 1 to 20 carbon atoms and may be straight chained or branched. An alkyl group is an optionally substituted straight, branched or cyclic saturated hydrocarbon group. When substituted, alkyl groups may be substituted with up to four substituent groups, at any available point of attachment. When the alkyl group is said to be substituted with an alkyl group, this is used interchangeably with “branched alkyl group”. Exemplary unsubstituted such groups include methyl, ethyl, propyl, isopropyl, a-butyl, isobutyl, pentyl, hexyl, isohexyl, 4,4-dimethylpentyl, octyl, 2,2,4-trimethylpentyl, nonyl, decyl, undecyl, dodecyl, and the like. Exemplary substituents may include but are not limited to one or more of the following groups: halo (such as F, Cl, Br, I), Haloalkyl (such as CC13 or CF3), alkoxy, alkylthio, hydroxyl, carboxy (—COOH), alkyloxycarbonyl (—C(O)R), alkylcarbonyloxy (—OCOR), amino (—NH2), carbamoyl (—NHCOOR— or —OCONHR), urea (—NHCONHR—) or thiol (—SH). Alkyl groups as defined may also comprise one or more carbon double bonds or one or more carbon to carbon triple bonds. “Lower alkyl” means an alkyl group, as defined below, but having from one to ten carbons, more preferable from one to six carbon atoms (eg. “C—C-alkyl”) in its backbone structure.

“Lower alkoxy” refers to O-alkyl groups, wherein alkyl is as defined hereinabove. The alkoxy group is bonded to the core compound through the oxygen bridge. The alkoxy group may be straight-chained or branched; although the straight-chain is preferred. Examples include methoxy, ethyloxy, propoxy, butyloxy, t-butyloxy, i-propoxy, and the like. Preferred alkoxy groups contain 1-4 carbon atoms, especially preferred alkoxy groups contain 1-3 carbon atoms. The most preferred alkoxy group is methoxy.

The terms “alkyl”, “cycloalkyl”, “heterocycloalkyl”, “cycloalkylalkyl”, “aryl”, “acyl”, “aromatic polycycle”, “heteroaryl”, “arylalkyl”, “heteroarylalkyl”, “amino acyl”, “non-aromatic polycycle”, “mixed aryl and non-aryl polycycle”, “polyheteroaryl”, “non-aromatic polyheterocyclic”, “mixed aryl and non-aryl polyheterocycles”, “amino”, and “sulphonyl” are defined in U.S. Pat. No. 6,552,065, Column 4, line 52 to Column 7, line 39.

The invention also relates to a pharmaceutically acceptable salt of hybrid.

The hybrid of the invention may also include a counterion. Generally, a counterion is required when the oligomer comprises a phosphate backbone. The terms “salt” and “counter ion” designate a pharmaceutically acceptable salts/counter ions and can include acid addition salts such as the hydrochlorides, hydrobromides, phosphates, nitrates, sulphates, hydrogen sulphates, alkylsulphates, arylsulphonates, acetates, benzoates, citrates, gluconates, maleates, fumarates, succinates, lactates, and tartrates; alkali metal cations such as Na, K, Li; alkali earth metal salts such as Mg or Ca; or organic amine salts. Exemplary organic amine salts are tromethamine (TRIS) salts and amino acid salts (e.g. histidine salts) of the compounds of the invention.

The term “intercalator” refers to a small, planar, aromatic molecule with the ability to insert between adjacent DNA base-pairs and participate in pi-pi stacking. Intercalators are often used as fluorescent probes to visualize DNA and drug-DNA interactions. Exemplary intercalators include 1-naphthylamine, 9-aminoanthracene, phenanthrene-9-amine, 1-aminopyrene, 6-aminochrysene, 1-naphtoic acid, anthracene, quinoline, acridine, phenanthridine, phenanthroline, ethidium bromide, and thiazole orange. Intercalators are described in detail in the literature, for example by Biebricher, A., Heller, I., Roijmans, R. et al. The impact of DNA intercalators on DNA and DNA-processing enzymes elucidated through force-dependent binding kinetics. Nat Commun 6, 7304 (2015). https://doi.org/10.1038/ncomms8304.

The term “pharmaceutically acceptable carriers” includes, but is not limited to, 0.01-0.1 M and preferably 0.05 M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments, the carrier can be a) 10% PEG (polyethylene glycol) 400 (v/v)+30% (v/v) HPβCD (hydroxypropyl β-cyclodextrin), 50% w/v+60% (v/V) Sterile Water for Injection or b) 0.1% (v/v) Tween 80+0.5% (w/v) carboxymethylcellulose in water. The carrier may comprise or provide a counterion.

The term “subject” refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. The subject can be, for example, a child, such as an adolescent, or an adult.

The term “treatment” refers to any treatment of a pathologic condition in a subject, such as a mammal, particularly a human, and includes: (i) preventing and/or reducing the risk of a pathologic condition from occurring in a subject which may be predisposed to the condition but has not yet been diagnosed with the condition and, accordingly, the treatment constitutes prophylactic treatment for the disease condition; (ii) inhibiting and/or reducing the speed of development of the pathologic condition, e.g., arresting its development; (iii) relieving the pathologic condition, e.g., causing regression of the pathologic condition; or (iv) relieving the conditions mediated by the pathologic condition and/or symptoms of the pathologic condition. Treatment of subjects who have previously and/or are currently, and/or are about to receive a cancer therapy are contemplated herein.

The term “therapeutically effective amount” refers to that amount of a compound of the invention that is sufficient to effect treatment, when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

In this specification, the term “cancer” should be taken to mean a cancer selected from the group consisting of: fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias. In a preferred embodiment, the cancer is selected from the group comprising: breast; cervical; prostate; ovarian, colorectal, lung, lymphoma, and leukemias, and/or their metastases.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Materials and Methods

Chemicals, reagents and HPLC grade solvents were sourced from Sigma Aldrich (Ireland) Ltd., unless otherwise stated, and were used without any further purification. Dichloromethane (DCM) was distilled from calcium hydride and stored under argon. All other solvents were used as supplied.

Note: Organic azides are hazardous materials and are known to be heat- and shock-sensitive. Explosive decomposition can potentially occur with very little energy input. To ensure safe manipulation and non-explosiveness for organic azides, the rule is that the number of nitrogen atoms must not exceed that of carbon, where (N_{C}+N_{O})/N_{N}≥3. (N=number of atoms). 4 is a potentially hazardous material, however all relevant safety precautions were undertaken while working with the material.

### 1.1: General Synthesis

¹H-NMR (600 MHz), ¹³C-NMR (151 MHz) and ¹⁹⁵Pt-NMR (129 MHz) spectra were obtained on a Bruker AC 600 MHz NMR Spectrometer. All experiments were performed at room temperature in either CDCl₃, DMSO-d₆ or DMF-d₇ were indicated. Chemical shift signals (δ) are given in parts per million (ppm) using the residual proton signals in the indicated solvents as internal standards. Coupling constants (J) are quoted in hertz (Hz). Signal peak multiplicities are assigned with the following: singlet(s), doublet (d), doublet of doublets (dd), doublet of doublet of doublets (ddd), triplet (t), doublet of triplets (dt), quartet (q), quintuplet (qn) and multiplet (m). Experimental spectra were analysed using MestReNova software (v. 14.2.0-26256, Mestrelab Research S.L.) FT-IR spectra were obtained from neat solids on a Perkin Elmer Spectrum Two Spectrometer. Melting points were obtained on a Stanford Research Systems MPA100 Optimelt apparatus. ESI-MS analysis was performed on a MaXis HD ESI-QTOF mass spectrometer (Bruker Daltonik GmbH) with data processing performed using Compass Data Analysis software (v 4.3, Bruker Daltonik GmbH).

### 1.2 Synthesis

Solid supports, standard DNA phosphoramidites and all other reagents used in the synthesis were purchased from Sigma Aldrich. Modified phosphoramidites were purchased from Glen Research. Oligonucleotides (ODNs) were synthesised on a K&A Laborgeräte H-8-SE LNA,DNA/RNA synthesiser using the standard 1.0 μmol phosphoramidite cycle. Coupling efficiencies were monitored by the trityl cation conductivity monitoring facility and was >98% for all oligonucleotides. Standard monomers (A, G, C and T) were coupled for 35 s and non-standard monomers were coupled for 360 s. ODNs were deprotected and cleaved from the solid support using a concentrated ammonia solution for 1 h at r.t., followed by heating in sealed vials for 5 h at 55° C. ODNs were purified using reverse-phase HPLC on a Gilson HPLC system using a Luna 10 μM C8 100 Å 250×10 mm column. For standard alkyne-modified ODNs, the gradient was 10-45% buffer B over 20 min with flow rate of 4 mL/min (buffer A: 0.1 M triethylammonium acetate (TEAA), buffer B: 0.1 M TEAA with 50% MeCN). Fraction volumes were reduced by rotary evaporation prior to redissolution in water and desalted using NAP-10 gel filtration columns purchased from GE Healthcare. All ODNs were characterised by negative-mode HPLC-mass spectrometry using a Waters Xevo G2-XS QT mass spectrometer with an Acquity UPLC system equipped with an Acquity UPLC oligonucleotide BEH C18 column (particle size: 1.7 μm; pore size 130 Å; column dimensions: 2.1×50 mm). Data was deconvoluted and analysed using Waters Mass Lynx software. Oligonucleotide synthesis with modified nucleobases was performed as described above with some minor deviations. Standard monomers (A, G, C, and T) were coupled for 35 s and non-standard monomers were coupled for 360 s. ODNs with incorporated pdU additions and those that were labelled with thiazole orange (TO_{B6}) or fluorescein/cyanine-5 dyes were purified with ammonium acetate (NH₄OAc) with a gradient of 15-45% buffer B over 25 min, flow rate of 4 mL/min (buffer A: 0.1 M NH₄OAc, buffer B: 0.1 M NH₄OAc 50% MeCN).

### 1.3: Thiazole Orange (TO) TFO: TO-NHS Ester Labelling

In a total volume of approx. 350 μL, 2000 nmol (10 eq.) of TO_{B6} NHS ester in DMF was added to 200 nmol of oligonucleotide dissolved in carbonate buffer (NaHCO₃/Na₂CO₃, 0.5 M, pH 8.74). The solution was shaken at 20° C. for 2 h prior to desalting by NAP column and purified by reverse-phase HPLC utilising a C8 column with a gradient of 0.1 M NH₄OAc+MeCN (50%) (Buffer B) in 0.1 M NH₄OAc (Buffer A). ESI-MS analysis, as previously described, was used to determine the purity of the labelled oligonucleotide. For ODNs that contained two labelling sites, 20 eq. of TO_{B6} NHS ester was used. For TOTFOs that were clicked to N₃-platinum(II) complexes, no purification of the TO labelled TFO was performed. Purification was only performed after click conjugation to ensure optimum yield.

### 1.4. Thiazole Orange (TO) TFO: TO-COOH Labelling

In a total volume of approx. 150 μL DMF, 800 nmol (20 eq.) of TOB6 carboxylic acid, 800 nmol (20 eq.) of (2-(1H-benzotriazol-1-yl)-1,1,3,3-tetramethyluronium hexafluorophosphate (HBTU) and 2400 nmol (48 μL, 50 mM) (60 eq.) of 4-methylmorpholine was dissolved. This mixture was shaken at 25° C. for 30 min prior to addition to a solution of AA-TOTFO (40 nmol, 1 eq.) in 150 μL of carbonate buffer (NaHCO₃/Na₂CO₃, 0.1 M pH 9.2). The entire solution was incubated for approx. 6 h at 37° C. The solution was desalted using NAP-10 gel filtration columns and purified using an Amicon. Ultra 0.5 mL centrifugal column.

### 1.5: Platinum(II)-TFO Hybrid Generation Using Click Chemistry

In a total volume of 500 μL (50:50 H₂O:DMSO or DMF, complex dependent) 500 nmol of Pt(II)-azide complex was added to 50 nmol of the alkyne-modified TFO. The solutions were degassed with argon and 500 nmol of Cu-TBTA complex in 50:50 H₂O:DMSO was added prior to final addition of 1000 nmol of Na-L-ascorbate. The click solutions were further degassed and stirred at 25° C. for 3-6 h prior to desalting by elution through a NAP-10 column. For click reactions concerning the 5′-BCN(CEP II)-dC modified TFO, the reaction was performed as above without the use of Cu-TBTA or Na-L-ascorbate. The volume was reduced and the residue re-dissolved in H₂O before purification by gradient reverse-phase HPLC utilising a C8 column. The Pt(II)-TFO hybrid purity was analysed by HPLC-ESI-MS.

### 1.6: UV-Thermal Melting Studies

Oligonucleotides were quantified on a NanoDropND-1000 UV-Vis Spectrophotometer. Thermal melting studies were performed on a Varian Cary 100 UV-Visible Spectrophotometer equipped with a 6×6 Peltier multicell system with temperature controller in Starna Scientific black-walled quartz cuvettes of 10 mm path length and 100 μL sample volume. Experimental measurements were monitored at 260 nm using Cary WinUV thermal application software. The TFO/Pt(II)-TFO hybrid and duplex samples were combined in a 2.5:1 μM ratio and dissolved in triplex buffer—10 mM phosphate, 150 mM NaCl and 2 mM MgCl₂ (pH 6). Pt-N₃-Complex/duplex melting samples were prepared similarly. Thermal melting was recorded between 12-90° C. (0.5° C./min with 2 min hold). In total, 3 heating ramps were performed. TM was calculated as an average of the first derivative of sigmoidal non-linear regression analysis of the triplex melting curve using GraphPad Prism 8 software.

### 1.7: Fluorescent Thermal Melting

Cisplatin and duplex samples were combined in a 2.5:1 μM ratio and dissolved in triplex buffer—10 mM phosphate, 150 mM NaCl and 2 mM MgCl₂ (pH 6) prior to incubation at 37° C. for 48 hours. SYBR green I (1 mL, Roche) was added to each sample and the melting profile for the triplex was analysed on a LightCycler® 480 II (Roche). Samples were heated to 99° C. with 10 fluorescence measurements recorded per ° C. A plot of sample fluorescence versus temperature is obtained and the first negative derivative of the sample was calculated. Samples were analysed in triplicate and T_{M} was calculated as an average of the first negative derivative of the melting curve. Melting curves were analysed and graphed using GraphPad Prism 8 software.

### 1.8: Triplex Formation

In a total volume of 5 μL triplex buffer, duplex target (40 bp, 1 pmol) was treated with increasing concentrations of alkyne-modified TFO (2.5-50 eq.). Samples were incubated at 37° C. for 0-48 h prior to addition of 6× loading dye (Thermo Scientific) and loaded onto a 20% polyacrylamide gel (50 mM Tris acetate, 150 mM NaCl, 2 mM MgCl₂, pH 6). Electrophoresis was performed at 70 V for 240 mins in triplex running buffer (50 mM Tris acetate, pH 6). Polyacrylamide gels were post-stained with SybrGold and visualised and imaged on a Syngene G: Box Mini 9 gel documentation system.

### 1.9: Triplex-Mediated Crosslink Activity

Target duplex (57 bp, 1 pmol) and off-target sequence (40 bp, 1 pmol) were treated with increasing concentrations of platinum (II)-TFO hybrid (2.5-50 eq.) and incubated at 37° C. for up to 48 h. Electrophoresis and visualisation was performed as previously reported.

### 1.10: Reversal of Triplex Formation by Sodium Cyanide

Fluorescently labelled duplex target (57 bp, 1 pmol) was treated with alkyne-modified TFO (50 eq.) and separately with platinum(II)-TFO hybrid (50 eq.). Samples were incubated at 37° C. for 48 h prior to addition of increasing concentrations of NaCN solution (1,000-300,000 eq.). Combined samples were incubated at r.t. for 18 h and quenched with 30% glycerol solution. Electrophoresis was performed as before. Gel visualisation and imaging was performed on a Syngene G: Box Mini 9 gel documentation system with integral Cy5 and 6-FAM filters.

### 1.11: Crosslink Investigation by Denaturing PAGE

In a total volume of 5 μL, fluorescently labelled duplex sequences (21/17 bp, 21 bp, 1 pmol) were treated with platinum(II)-TFO hybrid (TFO-17A-C, 50 eq.) Samples were incubated at 37° C. for a minimum of 48 h prior to quenching with 2× denaturing loading dye. Samples were loaded onto 20% denaturing PAGE (1× TBE, 7.4 M urea, pH 8.2) and subjected to electrophoresis at 300 V for 60 mins in 1× TBE running buffer.

### 2: Synthesis of Azide-Functionalised Ligands and Platinum(II) Complexes

### Route 1: Synthesis of cis-[Pt(2-azidopropane-1,3-diamine)Cl₂]

### Synthesis of Di-tert-boc-2-hydroxy-1,3-diaminopropane (1)

Reaction protocol was adapted from the literature method as reported by Kane et al. [23] To a solution of 1,3-diamino-2-propanol (2.000 g, 22.19 mmol, 1 eq.) and triethylamine (4.49 g, 44.38 mmol, 2 eq.) in a 1:1 THF/H₂O mix (80 ml) stirring at 0° C. was added di-tert-butyl dicarbonate (12.11 g, 55.48 mmol, 2.5 eq.) in THF (40 ml). The resulting solution was stirred at r.t. overnight (approx. 18 h). THF was removed in vacuo and approx. 40 ml of distilled H₂O was added to the solution before extraction with EtOAc (3×50 ml). Organic layer was dried over MgSO₄ and solvent removed in vacuo to yield the desired product as a clear pale oil, which subsequently solidified to white solid. Yield: 5.617 g, 87%. ¹H NMR (600 MHz, CDCl₃): 5.14 (s, 2H, NH), 3.78-3.73 (m, 1H, CH), 3.31-3.14 (m, 4H, (CH₂)₂), 1.46 (s, 18H, Boc). ¹³C NMR (151 MHz, CDCl₃): 157.27 (C, carbonyl), 79.81 (C, Boc), 71.14 (CH), 43.57 (CH₂), 28.36 (CH₃, Boc). IR (ATR, cm⁻¹): 3490, 3369, 3313, 2983, 2933, 1681, 1659, 1524, 1274, 1253, 1164, 1084, 970, 865, 581. mp 91-93° C. Anal. Calcd for C₁₃H₂₆N₂O₅: C, 53.78; H, 9.03; N, 9.65. Found: C, 53.88; H, 9.19; N, 9.59.

### Synthesis of Di-tert-boc-2-methanesulfonyl-1,3-diaminopropane (2)

2 was synthesised, with slight adaptation, according to a procedure reported by Abd Karim et al. [24] Di-tert-boc-2-hydroxy-1,3-diaminopropane (5.000 g, 17.22 mmol, 1 eq.) and triethylamine (5.23 g, 51.66 mmol, 3 eq.) were dissolved in 80 ml anhydrous DCM and purged with argon for 20 mins. Methanesulfonyl chloride (3.95 g, 34.44 mmol, 2 eq.) was dissolved in approx. 8 ml anhydrous DCM and added dropwise to the degassed solution with stirring over 30 minutes at 0° C. The mixture was allowed to gradually warm to r.t. and stirred overnight (approx. 18 h). 70 ml DI water was added to the mixture to react with excess MsCl and stirred for 3 h. Organic layer was separated and washed sequentially with 100 ml 10% NaHCO₃ and 100 ml brine (×2). Organic layer was dried over MgSO₄ and solvent removed in vacuo to afford a pale yellow/beige solid and recrystallised using hexane, to yield white solid. Yield: 4.755 g, 75%. ¹H NMR (600 MHz, CDCl₃): 5.18 (t, J=6.6 Hz, 2H, NH), 4.66 (p, J=5.2 Hz, 1H, CH), 3.49 (ddd, J=14.8, 7.5, 4.5 Hz, 2H, CH₂), 3.30 (dt, J=14.9, 5.8 Hz, 2H, CH₂), 3.09 (s, 3H, CH₃), 1.44 (s, 18H, Boc). ¹³C NMR (151 MHz, CDCl₃): 156.47 (C, carbonyl), 80.19 (C, Boc), 79.20 (CH), 40.96 (CH₂), 38.31 (CH₃, OMs), 28.46 (CH₃, Boc). IR (ATR, cm⁻¹): 3448, 3379, 2976, 2937, 1704, 1516, 1345, 1251, 1172, 1116, 1045, 950, 917, 800, 529, 460. mp 138-140° C. Anal. Calcd for C₁₄H₂₈N₂O₇S: C, 45.64; H, 7.66; N, 7.60. Found: C, 45.44; H, 7.42; N, 7.31.

### Synthesis of Di-tert-boc-2-azido-1,3-diaminopropane (3)

3 was synthesised, with slight adaptation, according to a procedure reported by Veerendhar et al. [25] To a solution of di-tert-boc-2-methanesulfonyl-1,3-diaminopropane (2.000 g, 5.43 mmol, 1 eq.) in 30 ml anhydrous DMF, NaN₃ (1.412 g, 21.72 mmol, 4 eq.) was added and the mixture stirred at 80° C. for 18 h. The mixture was allowed to cool to ambient temperature, poured into approx. 50 ml DI water and further cooled at to 0° C. prior to filtering to obtain white solid product. Yield: 1.38 g, 81%. ¹H NMR (600 MHz, CDCl₃): 5.06 (s, 2H, NH), 3.64 (s, 1H, CH), 3.43-3.31 (m, 2H, CH₂), 3.18-3.07 (m, 2H, CH₂), 1.44 (s, 18H, Boc). ¹³C NMR (151 MHZ, CDCl₃): 156.20 (C, carbonyl), 79.91 (C, Boc), 60.92 (CH), 40.80 (CH₂), 28.35 (CH₃, Boc). IR (ATR, cm⁻¹): 3340, 2966, 2936, 2110 (N₃), 1680, 1517, 1363,1267, 1239, 1153, 963, 862, 635.

### Synthesis of 2-azidopropane-1,3-diamine dihydrochloride (4) [26]

4 was synthesised according to the literature method previously reported by Urankar et al. To a solution of di-tert-boc-2-azido-1,3-diaminopropane (0.400 g, 1.27 mmol, 1 eq.) in EtOAc (3 ml) was added aqueous 6 M HCl (approx. 1.5 ml) dropwise with stirring. The reaction mixture was stirred at 0° C. for 30 mins followed by stirring for approximately 18 h at r.t. before cooling at 5° C. overnight. The resulting white precipitate was filtered and washed several times with EtOAc to afford the product as a white crystalline solid. Yield: 0.221 g, 93%. ¹H NMR (600 MHz, DMSO-d₆): 8.38 (s, 6H, (NH₃)₂), 4.23 (tt, J=8.6, 4.0 Hz, 1H, CH), 3.15 (dd, J=13.5, 4.0 Hz, 2H, CH₂), 2.90 (dd, J=13.4, 8.9 Hz, 2H, CH₂). ¹³C-NMR (151 MHZ, DMSO-d₆): 57.70 (CH), 40.49 (CH₂). IR (ATR, cm⁻¹): 2946, 2851, 2127 (N₃), 1503, 1462, 1361, 1279, 1203, 1081, 957, 933, 606. mp 227-233° C.

### Synthesis of cis-[Pt(2-azidopropane-1,3-diamine)Cl₂] (5, Pt-N₃-Cis)

Reaction protocol was adjusted from the literature method as previously reported by Urankar et al. [26] 1,8-Diazabicyclo[5.4.0]undec-7-ene (DBU) (0.081 g, 0.53 mmol, 2 eq.) was added to a solution of 2-azidopropane-1,3-diamine dihydrochloride (0.050 g, 0.265 mmol, 1 eq.) in 0.7 ml anhydrous DMF with stirring. To this solution was added cis-[Pt(DMSO)₂Cl₂] (0.112 g, 0.265 mmol, 1 eq.) and the solution was stirred at r.t. for 3 days. Approximately 2 ml DI H₂O was added and the mixture was cooled at 5° C. overnight. A grey precipitate was collected by filtration, and the product washed with DI H₂O (2×2 ml). Yield: 0.027 g, 27%. ¹H NMR (600 MHz, DMF-d₇) δ 5.26-5.18 (d, J=45.3 Hz, 4H, NH₂), 4.19 (m, 1H, CH), 2.93 (m, 2H, CH₂), 2.85 (ddq, J=13.1, 6.7, 3.9 Hz, 2H, CH₂). ¹³C NMR (151 MHZ, DMF-d₇) δ 59.88 (CH), 46.54 (CH₂). ¹⁹⁵Pt NMR (129 MHz, DMF-d₇) δ-2272. IR (ATR, cm⁻¹): 3324, 3182, 3118, 2108 (N₃), 1586, 1258, 1172, 1027, 821. ESI-MS: m/z. 403.9 [M+Na]⁺, 784.9 [2M+Na]⁺. C₃H₉Cl₂N₅ ¹⁹⁵Pt⁺ [M+Na]⁺: 402.9781. Found: 403.9758.

### Route 2: Synthesis of cis-[Pt(2-azidopropane-1,3-diamine)(CBDCA)] and cis-[Pt(2-azidopropane-1,3-diamine)(Oxalate)]

### cis-[Pt(2-azidopropane-1,3-diamine)I2] (6) [26]

To a solution of K₂PtCl₄ (0.300 g, 0.723 mmol, 1 eq.) in approx. 8 ml DI H₂O was added KI (1.20 g, 7.23 mmol, 10 eq.) and the mixture was stirred for 2 h. A solution of 2-azidopropane-1,3-diamine dihydrochloride (0.0.136 g, 0.723 mmol, 1 eq.) in 3 ml aqueous NaOH (0.058 g, 1.45 mmol, 2 eq.) was then added dropwise to the stirring mixture. Subsequently, the entire mixture was then heated at 60° C. for 10 mins, cooled to 0° C. and filtered to obtain brown/tan solid product. Sequential washes with DI H₂O, MeOH and Et₂O (5×5 ml) followed by drying over vacuum afforded cis-[Pt(2-azidopropane-1,3-diamine)I₂] as a brown solid. Yield: 0.352 g, 86% (from K₂PtCl₄).

### cis-[Pt(2-azidopropane-1,3-diamine)(CBDCA)] (7, Pt-N₃-Carbo) [26]

7 was synthesised, with slight adaptation, according to the literature method previously reported by Urankar et al. [26] Cis-[Pt(2-azidopropane-1,3-diamine)I₂] (0.352 g, 0.624 mmol, 1 eq.) was suspended in approx. 30 ml DI H₂O and a 5 ml aqueous solution of AgNO₃ (0.212 g, 1.25 mmol, 2 eq.) was then added dropwise with stirring. The solution was stirred for 3 h before filtering through a 0.22 μm Millipore pad to remove AgI. To the filtrate was added 3 ml of an aqueous solution of cyclobutane-1,1-dicarboxylic acid (CBCDA) (0.090 g, 0.624 mmol, 1 eq.) and NaOH (0.050 g, 1.25 mmol, 2 eq.). The solution was stirred for approx. 18 h in the dark and filtered through a 0.22 μm Millipore pad prior to the removal of solvent to yield a crude grey residue. 5 ml DI H₂O was added to the residue and cooled at 5° C. before vacuum filtration, washed with 5×5 ml H₂O, MeOH and Et₂O and finally dried to obtain an off-white solid. Yield: 0.118 g, 36%₄ (from K₂PtCl₄). ¹H NMR (600 MHz, DMSO-d₆) δ 5.45 (s, 2H, NH₂), 5.28 (s, 2H, NH₂), 3.91 (s, 1H, CH), 2.65 (qn, 4H, (CH₂)₂), 2.53 (m, 2H, CH₂), 2.48 (m, 2H, CH₂), 1.64 (qn, 2H, CH₂). ¹³C NMR (151 MHz, DMSO-d₆): 177.89 (C, carbonyl), 59.16 (CH, N₃), 56.06 (C, CBDCA), 45.86 (CH₂, N₃), 31.04 (CH₂, CBDCA), 30.71 (CH₂, CBDCA), 15.45 (CH₂, CBDCA). ¹⁹⁵Pt NMR (129 MHz, DMSO-d₆) δ-1942. IR (ATR, cm⁻¹): 3217, 3092, 2938, 2120 (N₃), 1622, 1576, 1342, 1268, 1106, 1024, 900, 768. ESI-MS: m/z. 453.1 [M+H]⁺, 475.1 [M+Na]⁺. ESI-MS calc. for C₉H₁₅N₅O ¹⁹⁵Pt⁺ [M+H]⁺: 453.0850. Found: 453.0846.

### cis-[Pt(2-azidopropane-1,3-diamine)(Oxalate)] (8, Pt-N₃-Oxali)

Reaction protocol was performed as reported in (7) with only minor changes. After the removal of Agl, an aqueous solution (4 ml) of sodium oxalate (0.188 g, 1.40 mmol, 1 eq.) was added to the retained filtrate and the entire solution was stirred for approx. 18 h in the dark. The solution was filtered through a 0.45 μm Millipore pad to obtain a grey solid. Yield: 0.405 g, 72% (from K₂PtCl₄). 1H NMR (600 MHz, DMSO-d₆) δ 5.76 (dt, J=10.5, 4.5 Hz, 2H), 5.46 (dt, J=15.3, 5.1 Hz, 2H), 3.99 (m, 1H), 2.63-2.56 (m, 2H), 2.55-2.51 (m, 2H). ¹³C NMR (151 MHz, DMSO-d₆): 166.57, 58.89, 45.48. ¹⁹⁵Pt NMR (129 MHz, DMSO-d6) δ-1968. IR (ATR, cm−1): 3182, 3092, 2106, 1654, 1388, 1248, 1212, 818. ESI-MS: m/z. 399.0 [M+H]+, 421.0 [M+Na]+. ESI-MS calc. for C₅H₉N₅O₄¹⁹⁵Pt+ [M+H]+: 399.0380. Found: 399.0374.

TFO Sequences

| | | |
|---|---|---|
| TFO 1 | 5′-CTCTTTCCTTCCCTTCTTTCGCTTTCCTC- | SEQ |
| | 3′ | ID 1 |
| TFO 2 | 5′-XTCTTTCCTTCCCTTCTTTCGCTTTCCTC- | SEQ |
| | 3′ | ID 2 |
| TFO 3 | 5′-CTCTTTCCTTCCCTTCTTTCGCTTUCCTC- | SEQ |
| | 3′ | ID 3 |
| TFO 4 | 5′-CTTCCCTCTTTCCGCCTGTCC-3′ | SEQ |
| | | ID 4 |
| TFO 5 | 5′-CTTCCCTCTTUCCGCCTGTCC-3′ | SEQ |
| | | ID 5 |
| TFO 17A | 5′-UCTTTCCTTCCCTTCTT-3′ | SEQ |
| | | ID 6 |
| TFO 17B | 5′-TCTTTCCTTCCCTTCTU-3′ | SEQ |
| | | ID 7 |
| TFO 17C | 5′-TCTTTCCTUCCCTTCTT-3′ | SEQ |
| | | ID 8 |

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### References

[1] a X. Shen, D. R. Corey, Nucleic Acids Res. 2018, 46, 1584-1600; b S. T. Crooke, B. F. Baker, R. M. Crooke, X.-H. Liang, Nat. Rev. Drug Discov. 2021.
[2] K. R. Fox, Curr. Med. Chem. 2000, 7, 17-37.
[3] K. R. Fox, T. Brown, D. A. Rusling, DNA Recognition by Parallel Triplex Formation, 2018.
[4] a J.-S. Sun, T. Carestier, C. Hélène, Curr. Opin. Struct. Biol. 1996, 6, 327-333; b P. E. Nielsen, Annu. Rev. Biophys. Biomol. Struct. 1995, 24, 167-183.
[5] a M. Takasugi, A. Guendouz, M. Chassignol, J. L. Decout, J. Lhomme, N. T. Thuong, C. Helene, Proc. Natl. Acad. Sci. 1991, 88, 5602-5606; b P. A. Havre, E. J. Gunther, F. P. Gasparro, P. M. Glazer, Proc. Natl. Acad. Sci. 1993, 90, 7879-7883.
[6] D. Oh, Nucleic Acids Res. 1999, 27, 4734-4742.
[7] M. Kundu, F. Nagatsugi, A. Majumdar, P. S. Miller, M. M. Seidman, Nucleosides, Nucleotides & Nucleic Acids 2003, 22, 1927-1938.
[8] R. Besch, C. Giovannangeli, T. Schuh, C. Kammerbauer, K. Degitz, J. Mol. Biol. 2004, 341, 979-989.
a I. Dieter-Wurm, M. Sabat, B. Lippert, J. Am. Chem. Soc. 1992, 114, 357-359; b C. Colombier, B. Lippert, M. Leng, Nucleic Acids Res. 1996, 24, 4519-4524; c E. Bernal-Me'Ndez, J.-S. Sun, F. González-Vílchez, M. Leng, New. J. Chem. 1998, 22, 1479-1483; d E. S. Gruff, L. E. Orgel, Nucleic Acids Res. 1991, 19, 6849-6854.
[9] a M. A. Campbell, T. M. Mason, P. S. Miller, Can. J. Chem. 2007, 85, 241-248; b M. A. Campbell, P. S. Miller, J. Biol. Inorg. Chem. 2009, 14, 873-881; c M. A. Campbell, P. S. Miller, Bioconjug. Chem. 2009, 20, 2222-2230; d M. K. Graham, P.S. Miller, J. Biol. Inorg. Chem. 2012, 17, 1197-1208; e M. K. Graham, T. R. Brown, P. S. Miller, Biochemistry 2015, 54, 2270-2282.
[10] R. Manchanda, S. U. Dunham, S. J. Lippard, J. Am. Chem. Soc. 1996, 118, 5144-5145.
[11] D. Cech, J. Schliepe, U. Berghoff, B. Lippert, Angew. Chem. Int. Ed. 1996, 35, 646-648.
[12] a S. K. Sharma, L. W. Mclaughlin, J. Am. Chem. Soc. 2002, 124, 9658-9659; b S. K. Sharma, L. W. Mclaughlin, J. Inorg. Biochem. 2004, 98, 1570-1577.
[13] L. Gordon, Terpyridine-Platinum (II) Complexes, WO/2017148193
[14] R. Koretsu, K. Kijo, Y. Seiho, K. Zuitoku, K. Knechi, Organometallic Complexes, JP2004075622A
[15] S. Dhar, R. Pathak, V. Popik, C. Mcnitt, Platinum (IV) Compounds and Methods of Making and Using Same, WO/2015134599 A2
[16] a M. Takasugi, A. Guendouz, M. Chassignol, J. L. Decout, J. Lhomme, N. T. Thuong, C. Helene, Proc. Natl. Acad. Sci. U.S.A. 1991, 88, 5602-5606; b S. K. Sharma, L. W. Mclaughlin, J. Am. Chem. Soc. 2002, 124, 9658-9659; cM. K. Graham, P. S. Miller, J. Biol. Inorg. Chem. 2012, 17, 1197-1208.
[17] P. E. Nielsen, Curr. Med. Chem. 2001, 8 (5), 545-550.
[18] E. E. Swayze, A. M. Siwkowski, E. V. Wancewicz, M. T. Migawa, T. K. Wyrzykiewicz, G. Hung, B. P. Monia, C. F. Bennett, Nucleic Acids Res. 2007, 35 (2), 687-700.
[19] S. Cirak et al. Lancet. 2011, 378 (9791), 595-605.
[20] Vitravene Study Group. Am. J. Ophthalmol. 2002, 133, 467-474.
[21] M. E. Visser, J. L. Witztum, E. S. G. Stroes, J. J. P. Kastelein, Eur. Heart. J. 2012, 33 (12), 1451-1458.
[22] P. P. Seth, A. Siwkowski, C. R. Allerson, G. Vasquez, S. Lee, T. P. Prakash, G. Kinberger, M. T. Migawa, H. Gaus, B. Bhat, E. E. Swayze. Nucleic Acids Symp. Ser. 2008, 52 (1), 553-554.
[23] S. A. Kane, H. Sasaki, S. M. Hecht, J. Am. Chem. Soc. 1995, 117, 9107-9118.
[24] K. J. Abd Karim, S. Binauld, W. Scarano, M. H. Stenzel, Polym. Chem. 2013, 4, 5542-5554.
[25] A. Veerendhar, S. Cohen, J. Frant, C. T. Supuran, R. Reich, E. Breuer, Heteroat. Chem. 2015, 26, 257-269.
[26] D. Urankar, J. Košmrlj, Inorg. Chim. Acta. 2010, 363, 3817-3822.

1. A pharmaceutical composition comprising a cis-platinum(II)-oligomer hybrid and a pharmaceutically acceptable carrier, wherein the cis-platinum(II)-oligomer hybrid is generated by conjugating an azide modified cis-platinum(II) complex with an alkyne-modified monomer of an oligomer by azide-alkyne cycloaddition, in which the oligomer comprises at least 10 contiguous nucleobase-bearing monomers, in which the oligomer is a triplex-forming oligonucleotide, and in which the cis-platinum(II)oligomer hybrid comprises a cis-platinum(II) complex capable of platinum(II) crosslinking to nucleic acids.

2. (canceled)

3. (canceled)

4. The pharmaceutical composition of claim 1, in which the azide modified cis-platinum(II) complex is a compound of Formula (I): 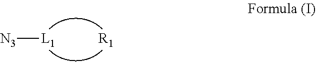
in which:
L₁ is a linker;
R₁ is a platinum containing DNA binding fragment of a cis-platinum (II) complex; and
L₁ binds to R₁ via bidentate coordination.

5. The pharmaceutical composition of claim 4, in which L₁ binds to R₁ via bidentate coordination selected from: diam(m)ine; disulphate; N,O; N,S; O,S; or O,O′ bidentate coordination.

6. The pharmaceutical composition of claim 1, in which the azide modified cis-platinum(II) complex is selected from:

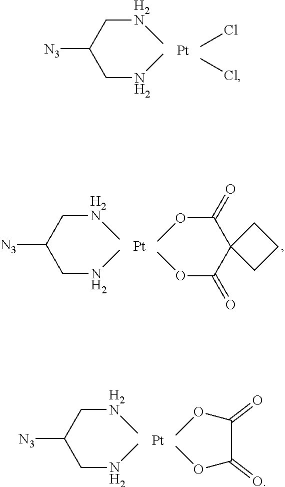

7. The pharmaceutical composition of claim 1, in which the alkyne modified monomer of the oligomer comprises an alkyne substituent conjugated to the nucleobase of the monomer.

8. The pharmaceutical composition of claim 1, in which the nucleobase of the alkyne modified monomer is selected from: 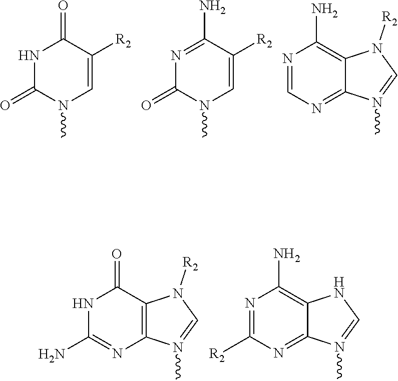
in which:
R₂ is selected from C(CH), L₂-C(CH) and a cycloalkyne substituent, in which L₂ is a linker.

9. The pharmaceutical composition of claim 1 in which the alkyne modified monomer of the oligonucleotide comprises an alkyne substituent conjugated to the 5′ phosphate of a ribose of the monomer.

10. The pharmaceutical composition of claim 7 in which the alkyne substituent is a cycloalkyne substituent selected from:

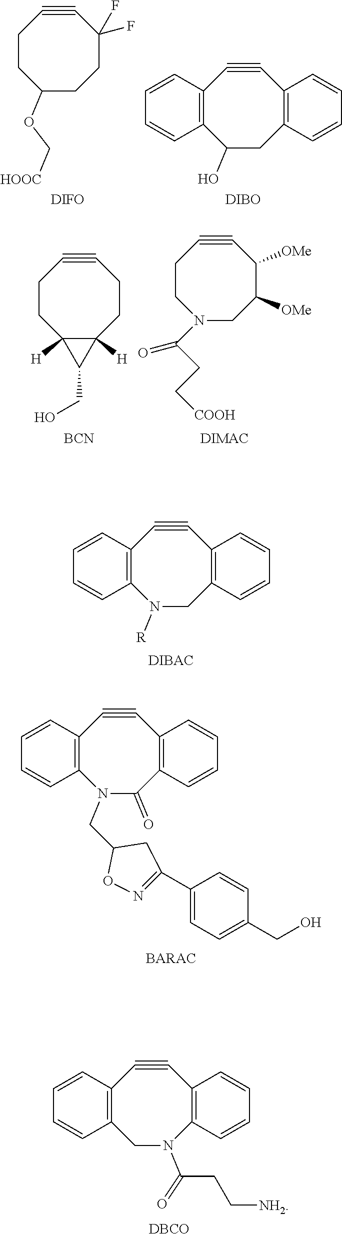

11. The pharmaceutical composition of claim 1, in which the azide alkyne cycloaddition is selected from metal-catalysed azide-alkyne cycloaddition and strain promoted azide-alkyne cycloaddition (SPAAC).

12. The pharmaceutical composition of claim 11, in which the metal catalysed azide-alkyne cycloaddition is copper(I) catalysed azide-alkyne cycloaddition (CuAAC).

13. (canceled)

14. (canceled)

15. (canceled)

16. The pharmaceutical composition of claim 1, in which the cis-platinum(II)-oligomer hybrid has a structure selected from Formula (II) or (III): 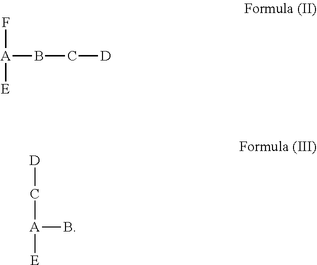
in which the oligomer is a triplex-forming oligonucleotide (TFO) comprising at least 10 contiguous nucleobase-bearing monomers, and in which:
A is a linker;
B is a purine or pyrimidine base;
A-B is a monomer of the TFO;
C is a linker comprising a triazole or bicyclic triazole group formed by alkyne-azide cycloaddition
D is a cis-platinum (II) complex capable of platinum (II) crosslinking to nucleic acids;
E is part of the TFO; and
F is absent or is part of the TFO.

17. The pharmaceutical composition of claim 16, in which A is a ribose unit.

18. (canceled)

19. (canceled)

20. The pharmaceutical composition of claim 16, in which F-A-E in Formula (II) has a structure selected from Formula (IV) or (V) in which E and F are as defined previously:

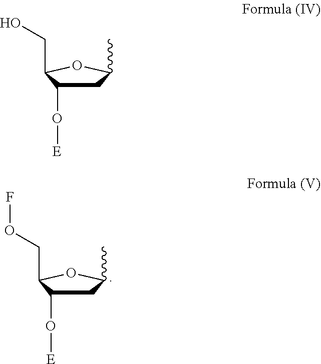

21-32. (canceled)

33. A method of treating cancer in a subject comprising administering a therapeutically effective amount of a pharmaceutical composition of claim 1 to the subject.

34. A method of making a cis-platinum(II)-oligomer hybrid comprising conjugating an azide modified cis-platinum(II) complex with an alkyne-modified monomer of an oligomer by azide-alkyne cycloaddition, in which the oligomer is a triplex-forming oligonucleotide comprising at least 10 contiguous nucleobase-bearing monomers, and in which the cis-platinum(II)-oligomer hybrid comprises a cis-platinum(II) complex capable of platinum(II) crosslinking to nucleic acids.


SEQUENCE LISTING (19 sequences)

SEQ ID NO: 1 - DNA, 29 bp, synthetic construct
  source 1..29: mol_type = other DNA
  modified_base 1: mod_base = OTHER; note = 5-prime-octadinyl-dC
ctctttccttcccttctttcgctttcctc

SEQ ID NO: 2 - DNA, 29 bp, synthetic construct
  source 1..29: mol_type = other DNA
  modified_base 1: mod_base = OTHER; note = 5-prime-BCN(CEP II)-dC
ctctttccttcccttctttcgctttcctc

SEQ ID NO: 3 - DNA, 29 bp, synthetic construct
  source 1..29: mol_type = other DNA
  modified_base 25: mod_base = OTHER; note = Int-octadinyl-dU
ctctttccttcccttctttcgctttcctc

SEQ ID NO: 4 - DNA, 21 bp, synthetic construct
  source 1..21: mol_type = other DNA
  modified_base 1: mod_base = OTHER; note = 5-prime-octadinyl-dC
cttccctctttccgcctgtcc

SEQ ID NO: 5 - DNA, 21 bp, synthetic construct
  source 1..21: mol_type = other DNA
  modified_base 11: mod_base = OTHER; note = Int-octadinyl-dU
cttccctctttccgcctgtcc

SEQ ID NO: 6 - DNA, 17 bp, synthetic construct
  source 1..17: mol_type = other DNA
  modified_base 1: mod_base = OTHER; note = Int-octadinyl-dU
tctttccttcccttctt

SEQ ID NO: 7 - DNA, 17 bp, synthetic construct
  source 1..17: mol_type = other DNA
  modified_base 17: mod_base = OTHER; note = Int-octadinyl-dU
tctttccttcccttctt

SEQ ID NO: 8 - DNA, 17 bp, synthetic construct
  source 1..17: mol_type = other DNA
  modified_base 9: mod_base = OTHER; note = Int-octadinyl-dU
tctttccttcccttctt

SEQ ID NO: 9 - DNA, 40 bp, synthetic construct
  source 1..40: mol_type = other DNA
accgtggcgagaaaggaagggaagaaagcgaaaggagcgg

SEQ ID NO: 10 - DNA, 40 bp, synthetic construct
  source 1..40: mol_type = other DNA
ccgctcctttcgctttcttcccttcctttctcgccacggt

SEQ ID NO: 11 - DNA, 32 bp, synthetic construct
  source 1..32: mol_type = other DNA
cgcttcccgaagggagaaaggcggacaggtat

SEQ ID NO: 12 - DNA, 32 bp, synthetic construct
  source 1..32: mol_type = other DNA
atacctgtccgcctttctcccttcgggaagcg

SEQ ID NO: 13 - DNA, 57 bp, synthetic construct
  source 1..57: mol_type = other DNA
aagccggcgaacgtggcgagaaaggaagggaagaaagcgaaaggagcgggcgctagg

SEQ ID NO: 14 - DNA, 57 bp, synthetic construct
  source 1..57: mol_type = other DNA
cctagcgcccgctcctttcgctttcttcccttcctttctcgccacgttcgccggctt

SEQ ID NO: 15 - DNA, 35 bp, synthetic construct
  source 1..35: mol_type = other DNA
ccgtggcgagaaaggaagggaagaaagcgaaagga

SEQ ID NO: 16 - DNA, 35 bp, synthetic construct
  source 1..35: mol_type = other DNA
tcctttcgctttcttcccttcctttctcgccacgg

SEQ ID NO: 17 - DNA, 21 bp, synthetic construct
  source 1..21: mol_type = other DNA
ggagaaaggaagggaagaagg

SEQ ID NO: 18 - DNA, 21 bp, synthetic construct
  source 1..21: mol_type = other DNA
ccttcttcccttcctttctcc

SEQ ID NO: 19 - DNA, 17 bp, synthetic construct
  source 1..17: mol_type = other DNA
ttcttcccttcctttct